(19) United States
(12) Patent Application Publication
(10) Pub. No.: US 2024/0049984 A1
(43) Pub. Date: Feb. 15, 2024

(54) REPLACEABLE CARTRIDGE FOR BREATHALYZER

(71) Applicant: BREATHABLOK LIMITED, London, Greater London (GB)

(72) Inventor: Alan John SULLIVAN, Durban (ZA)

(21) Appl. No.: 18/258,184

(22) PCT Filed: Dec. 17, 2021
(86) PCT No.: PCT/IB2021/061910

§ 371 Date: Jun. 16, 2023

(30) Foreign Application Priority Data
Dec. 18, 2020 (NL) 2027159

(51) Int. Cl. A61B 5/097; A61B 5/08

(52) U.S. Cl. CPC A61B 5/097; A61B 5/082; A61B 2562/166

(57) ABSTRACT

A breathalyser comprises a breath chamber 12 defining an inlet 14 for a breath sample and an outlet 16. A sensor 18 for an intoxicating substance is in fluid flow communication with the outlet 16. The sensor comprises an output for an electronic signal representative of a concentration of the intoxicating substance in the sample. A pump 20 moves the sample via the outlet 16 to the sensor 18. First electronic circuitry 22 comprises a controller 24 comprising a processor for processing the signal and to generate data relating to the concentration of the intoxicating substance. Second electronic circuitry 26 comprises a memory arrangement 28 which is in signal communication with the first electronic circuitry and stores the generated data. The first electronic circuitry 22 is permanently mounted in a main housing 30 of the breathalyser. At least the sensor 18 and the second electronic circuitry 26 form part of a cartridge 32.2 which is removably mountable on the main housing 30. The second electronic circuitry 26 is removably connectable to the first electronic circuitry 22.

### INTRODUCTION AND BACKGROUND

This invention relates to an interlock for a vehicle and more particularly to a breathalyser for an interlock for a vehicle.

An alcohol interlock for a vehicle typically comprises a breathalyser and a vehicle blocking device, which is connected to or otherwise in signal communication with the breathalyser. The interlock acts as a vehicle immobiliser which, when installed, can be brought into a not-blocking state (state in which the alcohol interlock does not immobilise the vehicle) only after presentation to and analysis by the breathalyser of a breath sample of a prospective driver with an alcohol concentration below a limit value.

Many known breathalysers comprise a fuel cell to detect the breath alcohol concentration from the sample. It is well known that performance of the fuel cell deteriorates over time. Furthermore, in use, breath samples contain moisture and droplets of mucus, alcohol and other condensation. This contaminates the fuel cell causing poor reading results and the fuel cell may require replacement or re-calibration. Information and frequency of these replacements may not be recorded and if replaced, there is no longer a historical knowledge base of past readings available. Most failures of interlocks are due to the fuel cell itself. The replacement is a complex process which involves opening the breathalyser body to remove the current fuel cell and to replace it with a new one. Highly skilled personnel must perform this task, because proper calibration is required and complete records must be kept of such replacements. For example, after replacement of a fuel cell, unless it could be proven that the new fuel cell was properly calibrated upon installation or replacement, the interlock manufacturer may be held liable in a case where the interlock is used and by some unfortunate event, a fatality due to drunk driving occurs. Furthermore, interlocks may in future become compulsory in many jurisdictions in all vehicles, which further drives the need for a simple and reliable means of replacing and calibrating fuel cells, whilst maintaining historical test readings and other records in a non-repudiable manner.

The applicant has also identified problems in the art to determine and monitor an effective and reliable operational life of the fuel cells that are being used in the breathalyser art. Merely adding the number of excitations (the number of times the fuel cell is used with a substance sample), in use, of a fuel cell and comparing the sum to an empirically determined limit value, may not be sufficient for at least some applications. As the fuel cell charges and discharges during excitations over time, an available voltage level between terminals of the fuel cell also drops over time. At some point, the voltage level becomes too low to be useful, and an end voltage level is reached, perhaps prematurely. At this point, the fuel cell should be replaced. Furthermore, the applicant believes that a fuel cell of a breathalyser, for example, in a vehicle of a heavy drinker or abuser of alcohol would “work harder” than one in a breathalyser in a vehicle of a normal or average user of alcohol. The effective and reliable operational life of a fuel cell in the former case is expected to be shorter or may require recalibration or replacement at shorter intervals.

### OBJECT OF THE INVENTION

Accordingly, it is an object of the present invention to provide a breathalyser with which the applicant believes the aforementioned disadvantages may at least be alleviated or which may provide a useful alternative for the known breathalysers.

### SUMMARY OF THE INVENTION

According to the invention there is provided a breathalyser comprising:
a breath chamber defining an inlet for a breath sample and an outlet;
a sensor for an intoxicating substance which is in fluid flow communication with the outlet and which comprises an output for an electronic signal representative of a concentration of the intoxicating substance in the sample;
fluid moving means for moving the sample via the outlet to the sensor;
first electronic circuitry comprising a controller comprising a processor for processing the signal and to generate data relating to the concentration of the intoxicating substance; and
second electronic circuitry comprising a memory arrangement which is in signal communication with the first electronic circuitry and for storing the generated data
wherein the first electronic circuitry is permanently mounted in a main housing of the breathalyser and wherein at least the sensor and the second electronic circuitry form part of a module which is removably mountable in or on the main housing and wherein the second electronic circuitry is removably connectable to the first electronic circuitry.

The breathalyser may form part of a human operable machine interlock. The machine may be a vehicle and the human may be a prospective driver.

The intoxicating substance may be alcohol, cannabis, marijuana or any other substance that may impair the human operator's ability to operate the machine.

The first electronic circuitry may comprise a first connector part and the second electronic circuitry may comprise a second connector part which is removably connectable to the first connector part.

In one example embodiment, the first electronic circuitry may be provided on a first or “mother” printed circuit (PC) board which is permanently mounted in the main housing and the module may comprise a second or “daughter” PC board for the second electronic circuitry, the second PC board may be removably receivable in the main housing and the second electronic circuitry may be removably connectable to the first electronic circuitry via the first and second connector parts.

In another example embodiment, the module may be in the form of a cartridge comprising a cartridge housing carrying the breath chamber, the sensor, the fluid moving means, the second electronic circuitry comprising the memory arrangement and the second connector part, the cartridge housing may be one of a) removably receivable in a slot defined in the main housing and b) removably attachable to the main housing in piggyback fashion and the second electronic circuitry may be removably connectable to the first electronic circuitry via the first and second connector parts.

The fluid moving means may comprise a pump preferably a suction pump.

The suction pump may comprise a bellows which is operable by a solenoid, which is controlled by the controller.

The memory arrangement of the second electronic circuitry is preferably a secure memory arrangement enabling confidential non-volatile data storage.

The fuel cell may comprise a fuel cell housing.

A temperature adjustment means may be provided adjacent, preferably immediately adjacent, at least part of the fuel cell housing.

A remainder of the fuel cell housing may be cladded by a thermal insulating layer or jacket.

The temperature adjustment means may comprise an electrically operable temperature adjustment means. The temperature adjustment means may be connected to the controller of the first electronic circuitry to be controlled by the controller. The temperature adjustment means may comprise a heater and/or a cooler. The means may comprise a Peltier module, for example.

The electronic signal may be in the form of a curve, which after excitation of the fuel cell by a breath sample, reaches a peak value and then decays to a base line value and which curve has at least some of the following parameters: i) a peak response value; ii) a peak response time, which is the time from excitation of the fuel cell to when the peak response is reached; iii) an entire response time, which is the time from excitation to when the signal again reaches the base line; and iv) a surface area under the curve, at least some of said parameters may vary over time as a function of number of excitations and the secure memory arrangement may be configured to store: a) pre-derived profile data, as a function of number of excitations, relating to at least some of the parameters; and b) a counter for a value for the number of past excitations of the fuel cell, in use.

Alternatively, or in addition, the electronic signal may be in the form of a curve, which after excitation of the fuel cell by a breath sample, reaches a peak value and then decays to a base line value, the controller of the first electronic circuitry may be configured to process the signal to derive data relating to electrical charge which is caused by each excitation of the fuel cell and to store the data in the memory arrangement of the second electronic circuitry.

The processing may comprise taking samples of the curved signal over time, determining a mathematical integral of the signal and converting the integral to data relating to electrical charge in coulomb.

In some embodiments the intoxicating substance may be alcohol, the concentration of the intoxicating substance may be breath alcohol concentration (BrAC) and the sensor may comprise an alcohol fuel cell.

The processor of the controller of the first circuitry may execute an algorithm which utilizes the value in the counter and the pre-derived profile data to generate BrAC data for the sample, thereby taking into account variations in the parameters with number of excitations.

The BrAC data for each sample and associated time and date data may be stored by the controller in the secure memory arrangement.

In other embodiments, the intoxicating substance may be marijuana and the electronic signal at the output of the sensor may be representative of a concentration of tetrahydrocannabinol (THC) in the breath sample. In these embodiments the sensor may comprise carbon nanotubes.

The invention also extends to a replaceable module for a breathalyser comprising a main housing, the main housing accommodating at least first electronic circuitry comprising a controller comprising a processor for processing a signal representative of a concentration of an intoxicating substance in a breath sample in a breath chamber of the breathalyser and which signal is received from a sensor for an intoxicating substance which is in fluid flow communication with an outlet of the breath chamber, the processor, in use, generating data relating to the concentration of the intoxicating substance, the module comprising:
the sensor; and
second electronic circuitry comprising a memory arrangement;
wherein the module is removably mountable in or on the main housing and wherein the second electronic circuitry is removably connectable to the first electronic circuitry.

In a presently preferred form, the replaceable module may be in the form of a cartridge comprising a cartridge body, the cartridge body may carry at least the breath chamber defining an inlet for the breath sample and an outlet; the sensor; fluid moving means for moving the sample via the outlet to the sensor; and the second electronic circuitry comprising a second electrical connector part which is presented on an outside of the cartridge body, the cartridge body may be one of a) removably receivable in a slot defined in the main housing and b) removably attachable to the main housing in piggyback fashion and wherein the second electronic circuitry is removably connectable to the first electronic circuitry via the second electrical connector part engaging a first electrical connector part of the first electronic circuitry on the main housing.

### BRIEF DESCRIPTION OF THE ACCOMPANYING DIAGRAMS

The invention will now further be described, by way of example only, with reference to the accompanying diagrams wherein:

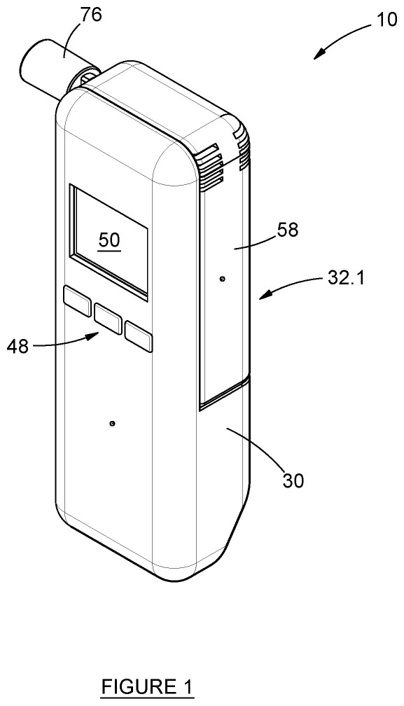

FIG. 1 is a diagrammatic representation of a first example embodiment of a breathalyser of a vehicle interlock;

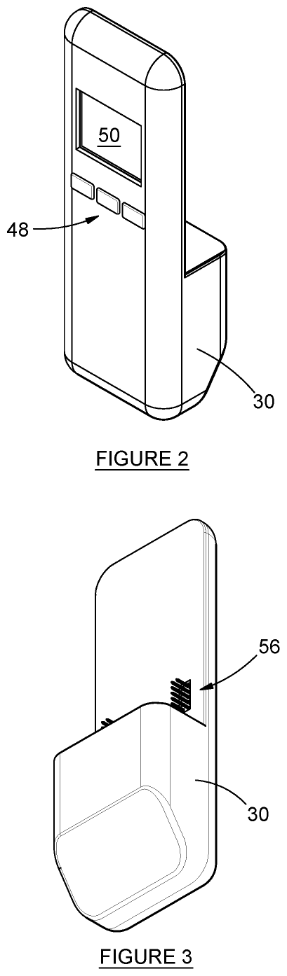

FIG. 2 is a first perspective view of a main housing of the breathalyser;

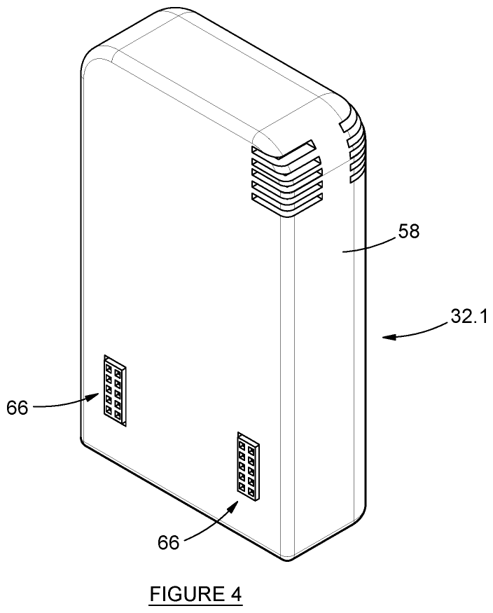

FIG. 3 is a second perspective view of the main housing presenting a first electrical connector part;

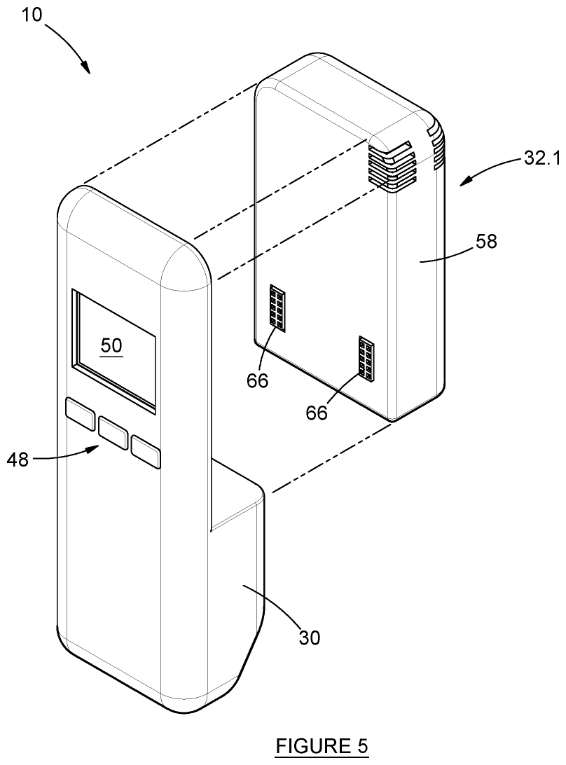

FIG. 4 is a perspective view of a replaceable cartridge of the breathalyser presenting a second electrical connector part;

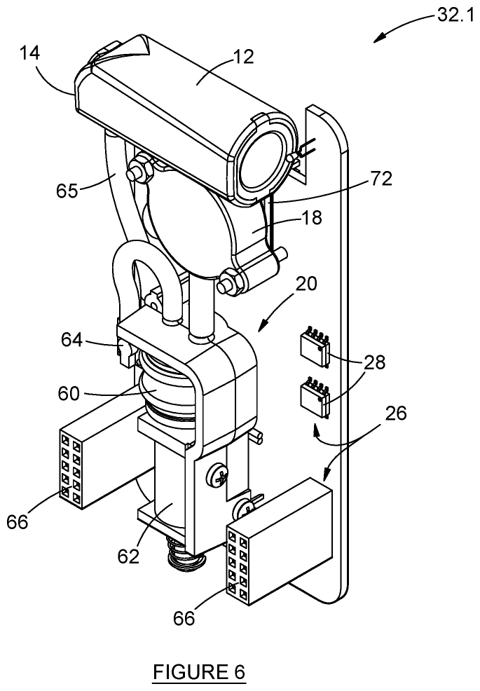

FIG. 5 is an exploded perspective view of the main housing and the cartridge;

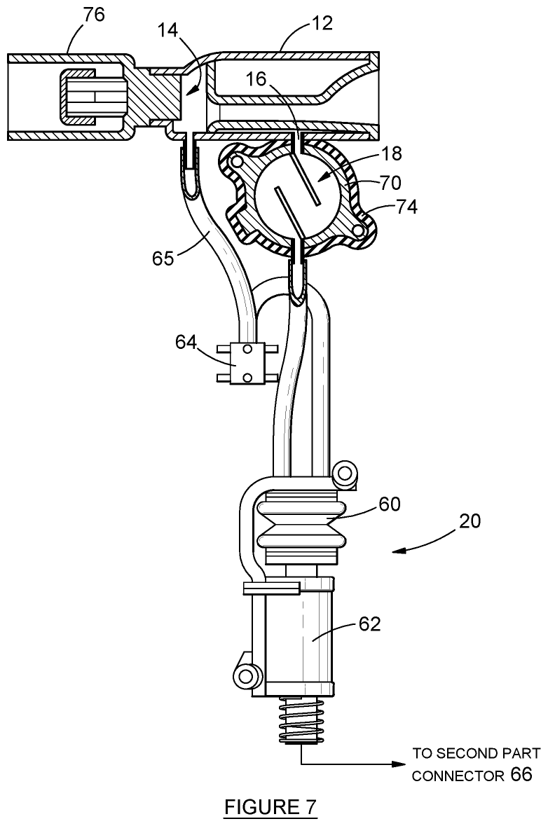

FIG. 6 is a perspective view of contents of the cartridge;

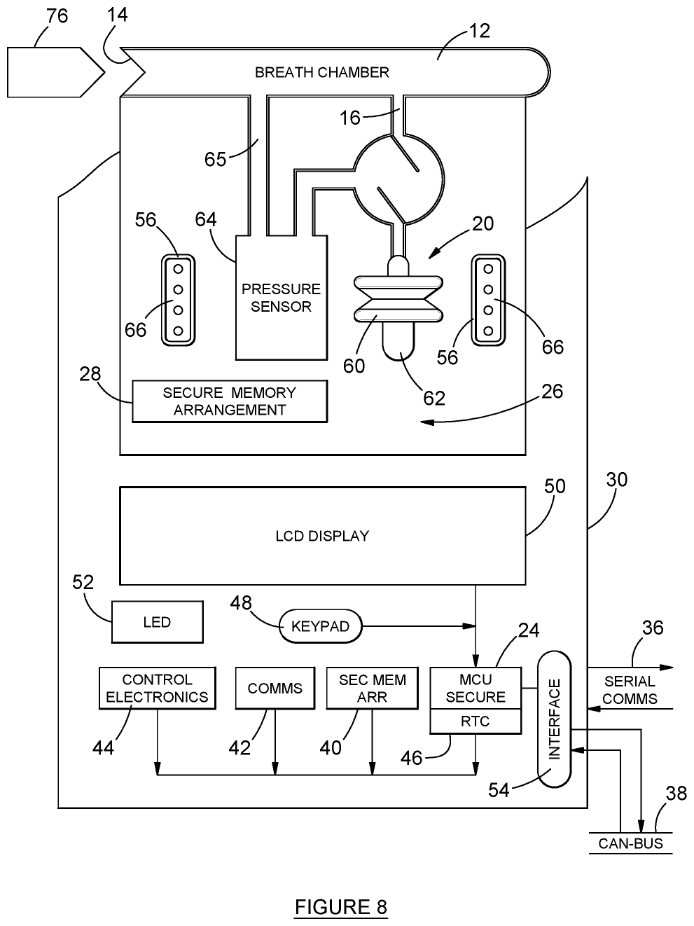

FIG. 7 is another view, partly in section, of the contents of the cartridge;

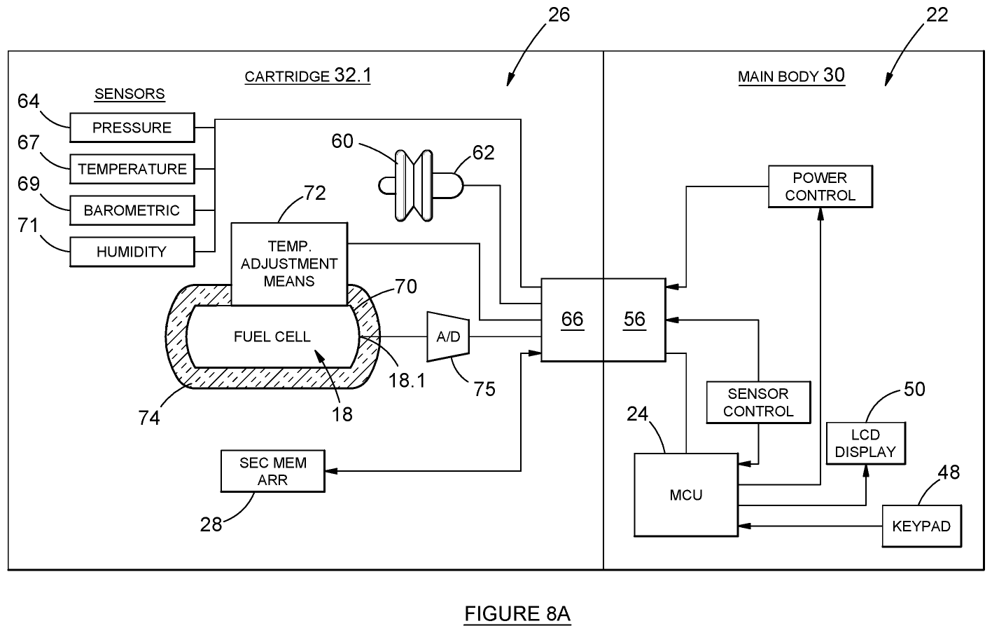

FIG. 8 is a high level block diagram of the breathalyser;

FIG. 8A is a more detailed block diagram of relevant parts of the breathalyser;

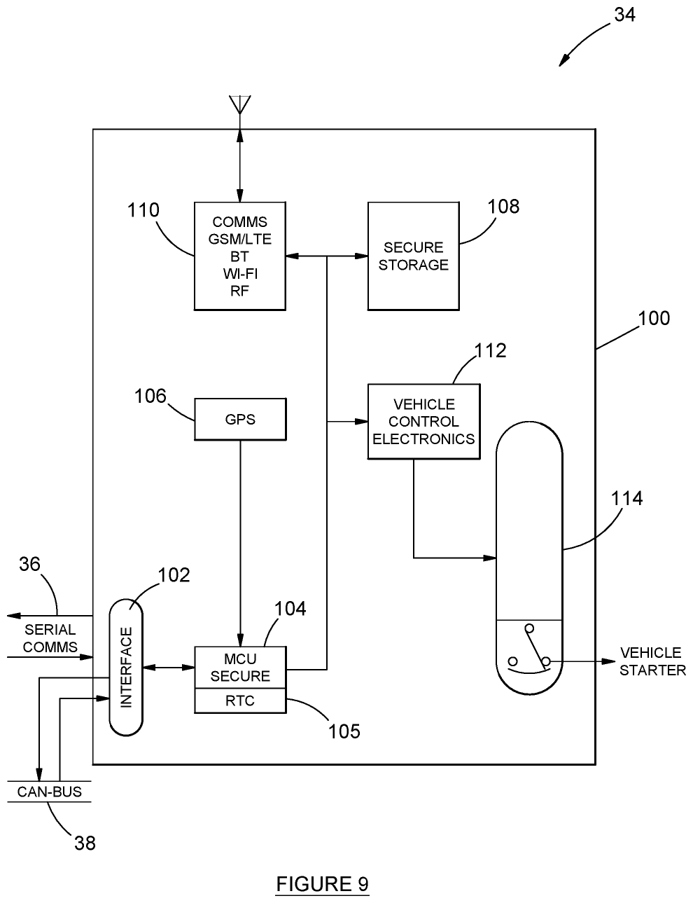

FIG. 9 is a block diagram of a blocking device of the interlock;

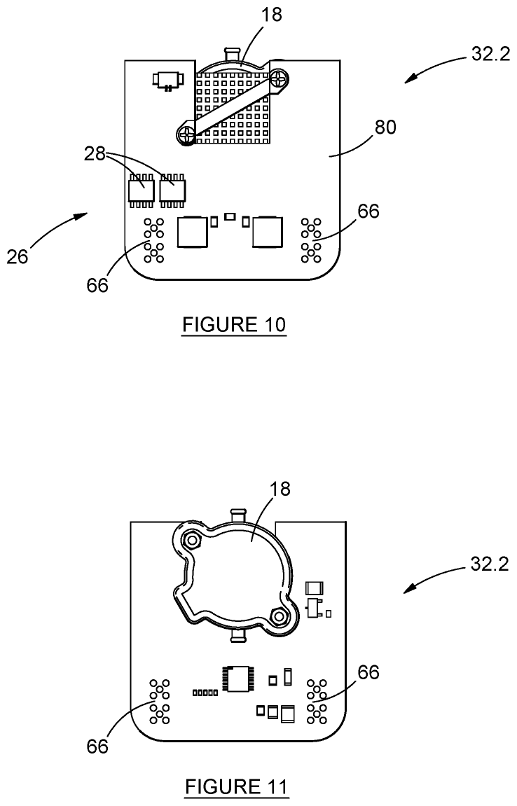

FIG. 10 is a first view of a second example embodiment of the module;

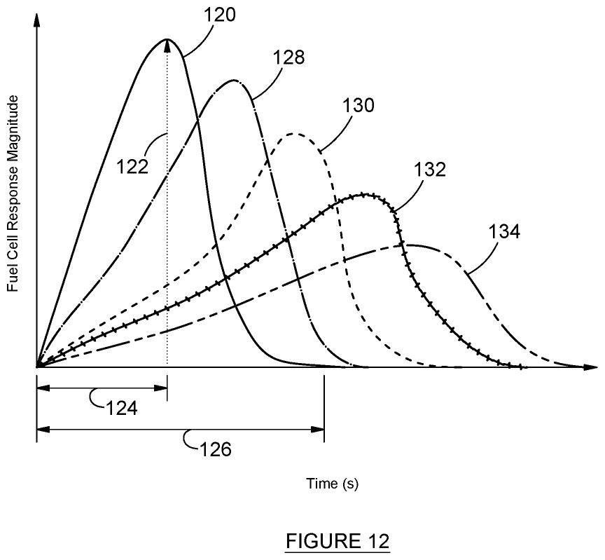

FIG. 11 is an opposite view of the second example embodiment of the module;

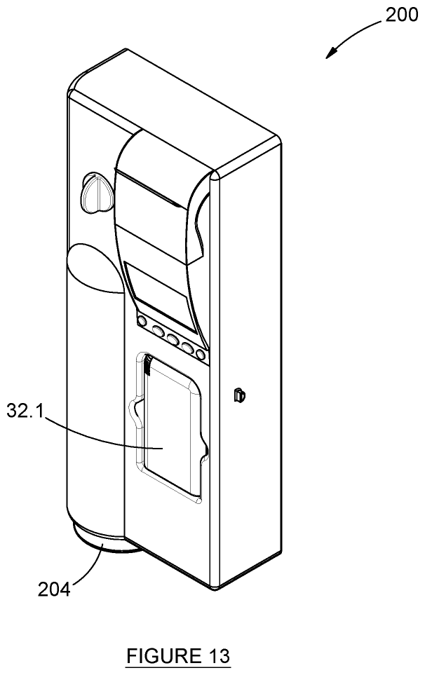

FIG. 12 is a graph of signal responses, of successive excitations over time, of a fuel cell forming part of the cartridge of the breathalyser;

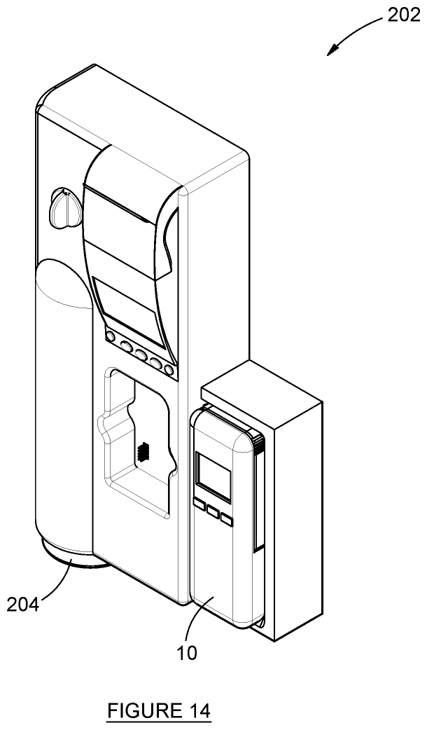

FIG. 13 is perspective view of an example embodiment of a calibration station for the cartridge; and

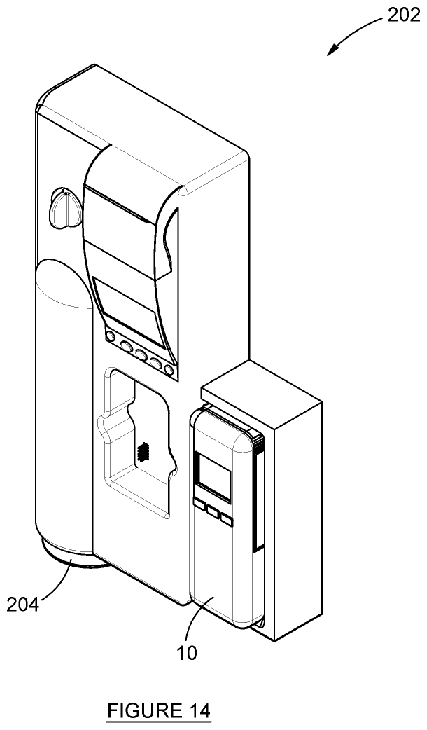

FIG. 14 is a perspective view of an example embodiment of a calibration station for the breathalyser.

### DESCRIPTION OF A PREFERRED EMBODIMENT OF THE INVENTION

An example embodiment of a breathalyser is generally designated by the reference numeral 10 in FIGS. 1, 5, 8 and 8A.

Referring to FIGS. 8 and 8A, the breathalyser comprises a breath chamber 12 defining an inlet 14 for a breath sample and an outlet 16. A sensor 18 for an intoxicating substance is in fluid flow communication with the outlet 16. The sensor comprises an output 18.1 (shown in FIG. 8A) for an electronic signal representative of a concentration of the intoxicating substance in the sample. A fluid moving means 20, in use and during a test, moves the sample via the outlet 16 to the sensor 18. First electronic circuitry 22 comprises a controller (MCU) 24 comprising a processor for processing the signal and to generate data relating to the concentration of the intoxicating substance in the sample. Second electronic circuitry 26 comprises a memory arrangement 28 which is in signal communication with the first electronic circuitry and stores the generated data. The first electronic circuitry 22 is permanently mounted in a main housing 30 of the breathalyser. At least the sensor 18 and the second electronic circuitry 26 form part of a module 32.1 (shown as a cartridge in FIGS. 1, 4, 5 and 6) or 32.2 (shown as a “daughter” printed circuit board in FIGS. 10 and 11) which is removably mountable on or in the main housing 30. The second electronic circuitry 26 is removably connectable to the first electronic circuitry 22, as will be described in more detail below.

The breathalyser 10 may form part of an intoxicating substance interlock for a human operable machine. The machine may be a vehicle and the human may be a prospective driver. The intoxicating substance may be alcohol, cannabis or any other substance that may impair the human operator's ability to operate the machine.

As an example, an alcohol interlock for a vehicle typically comprises a breathalyser 10 and a vehicle blocking device 34 (shown in FIG. 9). The breathalyser 10 is typically mounted in the passenger compartment (not shown) of the vehicle within easy and convenient reach of the driver. The blocking device 34 on the other hand is deep hidden on the body of the vehicle, so that it is not easily accessible by the driver, in order to avoid tampering with the blocking device. The breathalyser 10 is in signal communication with the blocking device 34 via a wireless or wired communication path 36 (shown in FIGS. 8 and 9) or via a CAN bus 38 of the vehicle. The interlock acts as a vehicle immobiliser which, when installed, can be brought into a not-blocking state (state in which the alcohol interlock does not immobilise the vehicle) only after presentation to and analysis by the breathalyser 10 of a breath sample of a prospective driver with an alcohol concentration below a limit value.

Referring firstly to the example embodiment of the breathalyser 10 in FIGS. 1 to 8A, the breathalyser comprises a main body 30. Referring in particular to FIG. 8, the first electronic circuitry 22 is permanently mounted in the main housing 30. The first circuitry 22 comprises the secure MCU 24, a secure memory arrangement 40, communications module(s) 42, control electronics 44, a real time clock 46, a keypad 48, an LCD display 50, a light emitting diode 52 and an interface 54 for communications with the blocking device 34. A special algorithm for computing an accurate substance concentration value runs on the secure MCU 24. The first circuitry 22 also comprises a first electrical connector part 56 which is presented on an outside of the main body 30, as best shown in FIG. 3.

In the above example embodiment, the module is in the form of a cartridge 32.1. The cartridge comprises a cartridge housing 58. Although a presently preferred form-factor for the cartridge housing 58 is shown in the figures, it will be appreciated that the cartridge housing 58 may have any other suitable or desired form-factor. The cartridge housing houses at least the second electronic circuitry 26, the breath chamber 12, the sensor 18 for an intoxicating substance which may comprise a fuel cell (and which will be described in more detail below) and the air moving means 20. The air moving means in this example embodiment comprises a bellows 60, a solenoid 62 and a pressure sensor 64. The fuel cell 18 is in fluid flow communication with the outlet 16 of the breath chamber 12 and the pressure sensor is also in fluid flow communication with the breath chamber 12.

The second circuitry 26 comprises at least a secure memory arrangement 28 and a second connector part 66. The secure memory arrangement 28 is configured to store initial fuel cell profile data (as described below), test data together with time and date data (also as described below) as well as a fuel cell excitation counter (also referred to below). The second connector part 66 is presented on an outside of the cartridge body 58, as best shown in FIG. 4. As shown in FIG. 8A, in a most preferred embodiment, the second circuitry 26 also comprises the pressure sensor 64, a temperature sensor 67, a barometric sensor 69 and a humidity sensor 71

As best shown in FIG. 5, the cartridge body 58 is removably mountable on the main body 30 and the first connector part 56 is removably connectable to the second connector part 66, so that the first electronic circuitry 22 and the second electronic circuitry 26 are in signal communication with one another. Hence, in use, when the fuel cell 18 needs replacement, a new cartridge body 58 is easily and conveniently mounted on the main body 30 in piggyback fashion with the first and second connector parts 66, 56 and hence the first and second electronic circuitry 22, 26 electrically connected to one another.

Referring to FIGS. 7 and 8A, the fuel cell typically comprises a fuel cell housing 70. A means 72 for adjusting (by making it higher or lower) temperature inside the fuel cell to a desired temperature (typically 35° C.) is provided immediately adjacent at least part of the housing. The means 72 may comprise an electrically operable heater element and/or cooler element. The means 72 may comprise a Peltier module, for example. The rest of the fuel cell housing 70 is cladded by a thermal insulating layer or jacket 74. The fuel cell 18 may be a breath alcohol sensor manufactured by Dart Sensors Limited.

Further as best shown in FIG. 8A, the output 18.1 of the fuel cell is connected to an analogue to digital (A/D) converter 75. The A/D converter also forms part of the second circuitry 26 and is connected to the second connector part 66.

A typical signal response at the output 18.1 of the fuel cell is shown at 120 in FIG. 12. The response curve comprises the following four important parameters: a peak response 122; a peak response time 124, which is the time from excitation of the fuel cell to when the peak response is reached; an entire response time 126, which is the time from excitation to when the signal again reaches a base line; and the area under the curve or integral under the curve. It is known that for fuel cells of the above kind, the peak response and area under the curve decrease due to ageing of the cell and number of excitations of the cell. It is also known that the peak response time and the entire response time increase over time and as a result of successive excitations for the above reasons. This is graphically illustrated at 120, 128, 130, 132 and 134 in FIG. 12.

As best shown in FIGS. 1, 7 and 8, a replaceable mouthpiece 76 is connectable to the inlet 14 of the breath chamber 12.

The MCU 24 of the first circuitry 22 may be a STMicroelectronics STM32F4+ Series device. The secure memory 28 of the second circuitry enables authentication and confidential non-volatile data storage and may, for example, comprise one or more devices manufactured and sold by MICROCHIP™ under the number ATAES132A 32K AES Serial EEPROM.

When the cartridge 32.1 is mounted on the main body 30 with the connector parts 56,66 connected as explained above, the secure memory arrangement 28 is connected to the secure MCU 24 by an Inter-IC bus such as a I2C bus (a type of bus designed by Philips Semiconductors) which is used to connect integrated circuits (IC's). I2C is a multi-master bus, which means that multiple chips can be connected to the same bus and each one can act as a master by initiating a data transfer. This allows at least two EEPROM devices of the secure memory arrangement 28 to be located in the cartridge 32.1 giving enough storage space for parameter requirements of the fuel cell profile (as will be described below), calibration information (date and time) relating to the fuel cell as well as for recording all data (date, time and measured breath alcohol concentration (BrAC)) of all breath tests performed using the fuel cell 18 on the cartridge 32.1.

It is envisaged to use block chain of all events and parameters to prevent any tampering with the resulting certificate. Blockchain is a system of recording information in a way that makes it difficult or impossible to change, hack, or cheat the system. A blockchain is essentially a digital ledger of transactions that is duplicated and distributed across an entire network of computer systems on the blockchain.

This ensures that the cartridge 32.1 provides a unique secure vault of all profiling and calibration information for purposes of CENELEC EN 50436 requirements and breath test data for possible future legal proceedings when it may be required to prove proper and correct calibration and operation of the fuel cell 18 in the cartridge 32.1.

In a second example embodiment of the breathalyser, the removable module 36.2 comprises a daughter printed circuit (PC) board 80 which is shown in FIGS. 10 and 11. The daughter PC board 80 hosts the second electronic circuitry 26, including the secure memory arrangement 28 and the second part connector 66, as well as the fuel cell 18 with associated temperature adjustment means 72. The daughter PC board 80 is removably receivable in the main housing of the breathalyser and is removably connectable to a mother PC board (not shown) which is permanently housed in the main housing. In this case, the breath chamber 12 and fluid moving means 20 are also permanently mounted in the main housing.

Reference is now made to FIG. 9 where the vehicle blocking device 34 is depicted. As stated above, the vehicle locking device 34 is in signal communication with the breathalyser 10 via the serial link 36, the CAN-bus or wirelessly. The blocking device comprises a housing 100 which is deep hidden on the body of the vehicle for the reasons mentioned above. The blocking device comprises an interface 102 cooperating with the interface 54 of the breathalyser 10. The interface 102 is connected to a secure controller 104 comprising a processor. The secure controller 104 is connected to a real time clock 105, a GPS receiver 106, a secure memory arrangement 108, a communications module(s) 110 and vehicle control electronics 112. The vehicle control electronics 112 is connected to a relay 114, for example, which is connectable to the vehicle starter, for example, to switch the vehicle from the blocking state to the non-blocking state, depending on whether the breath alcohol concentration in an accepted breath sample delivered by the prospective driver is below the breath alcohol concentration limit.

A supplier of cartridges 32.1 comprising the above fuel cells 18 would characterize the fuel cell performance by subjecting a plurality of the fuel cells to tests, to derive profile data relating to the performance of the fuel cells over time (typically a year or even three) as a function of excitations, more particularly relating to at least some of the above important parameters referred to in FIG. 12. Accelerated tests over a temperature range of −40 degrees C. to +85 degrees C. and various other performance criteria as set out in CENELEC EN 50436 (such as different concentrations of Ethanol) are used.

The profile data is stored as initial information in the secure memory arrangement 28 of the cartridge 32.1. The data is stored as a set of tables and constants which provides a lookup of anticipated values for the four parameters. The excitation counter is used to count every breath sample test or calibration. As each cartridge will operate in different environments, other individual fuel specific parameters, such as pressure, temperature, altitude and humidity (derived from sensors 64, 67, 69 and 71 respectively in FIG. 8A) will be used with the counter to dynamically adapt the algorithm running on the secure MCU 24 as the fuel cell ages, to always produce a highly accurate BrAC data reading. The BrAC data together with time and date data for the test are transmitted by the MCU 24 to the secure memory arrangement 28 where it is stored in secure manner, to provide a non-repudiable record of the sample and data.

It may be required to calibrate a new fuel cell before it is inserted into the breathalyser or after a period of use. To this end, calibration stations 200 and 202 depicted in FIGS. 13 and 14, respectively are provided. The station 200 in FIG. 13 is configured to receive the cartridge 32.1 only and the station in FIG. 14 is configured to receive the breathalyser 10. The station comprises a canister 204 for delivering into the breath chamber 12 a known concentration of alcohol using a regulated flow. The reading data is used to adjust the initial data should any deviation from initial calibration be detected and will be captured in the secure memory arrangement.

In an example embodiment, the breathalyser 10 comprises an accelerometer (not shown) or another suitable device for detecting motion and/or orientation in space of the breathalyser 10. The accelerometer is connected to the MCU 24 of the first electronic circuitry 22. The MCU 24 is also connected to the temperature adjustment means 72, which may comprise an electrically operable heater element. Since electrical power available to energize the heater element is normally severely limited, the MCU 24 may be configured, as soon as motion of the breathalyser 10 is detected by the accelerometer (for example when the breathalyser is removed form a cradle in the vehicle), to commence energizing the heating element, in order to get temperature at or of the fuel cell closer to a desired temperature, by the time a user blows into the breathalyser. Alternatively, and/or in addition, the accelerometer may be used by the MCU 24 to detect possible circumvention or unauthorized use of the breathalyser, for example by detecting unauthorized orientations of the breathalyser in space.

Regarding an effective and reliable operational life of the fuel cells referred to in the Introduction and Background of this specification, the number of times the fuel cell is used with a substance sample (number of excitations) may be used to give an indication of the cell performance over time and may give an indication when it may need to be replaced. However, it is believed that not only the number of excitations is relevant, but also the substance concentration in each sample. It is believed that electrical charge caused during excitation is proportional to the substance concentration. The higher the concentration, the more the electrical charge caused. The concentration during use therefore influences the expected operational life of the fuel cell. To enhance the above number of excitations metric, in an example embodiment of the breathalyser, data relating to electrical charge caused by each substance sample is also recorded, processed, typically in coulomb, and stored. The data is stored in the memory arrangement 28 of the second electronic circuitry 26 and a running total of charge in coulomb is also stored in the memory arrangement 28.

By taking many samples of fuel cell voltage over time and determining the integral, it is possible to calculate the charge caused by the substance sample. This value is then accumulated and stored in the memory arrangement 28 to be used to determine or predict, by not only the number of excitations but now also having a measure of the charge it has caused or produced, the operational life of the fuel cell. It is believed that a fuel cell may have a limited amount of coulomb that it could generate during an operational life and that the fuel cell should be replaced or at least recalibrated, before that amount is reached.

The above data may also be transferred and stored in the secure storage 108 of the vehicle blocking device 34. This data may then intermittently be transmitted via communications module 110 to a central backend for appropriate steps, if necessary.

The above data relating to charge caused, allows a manufacturer to make accurate assessment of the point at which the fuel cell should be replaced or recalibrated. This also will determine the warranty agreement that would be supplied with the cartridge with fair usage agreements.

Although the actual breathalyser is sold with typically a 1-year calibration certificate, use of the above data relating to the fuel cell may now be used with Al analysis, to indicate possible pre-mature failure. The ability to determine if the fuel cell is faulty and susceptible to erroneous reading is an invaluable feature to prevent potentially fatal accidents involving intoxicated drivers.

1. A breathalyser comprising:
a breath chamber defining an inlet for a breath sample and an outlet;
a sensor for an intoxicating substance which is in fluid flow communication with the outlet and which comprises an output for an electronic signal representative of a concentration of the intoxicating substance in the sample;
fluid moving means for moving the sample via the outlet to the sensor;
first electronic circuitry comprising a controller comprising a processor for processing the signal and to generate data relating to the concentration of the intoxicating substance; and
second electronic circuitry comprising a memory arrangement which is in signal communication with the first electronic circuitry and for storing the generated data wherein the first electronic circuitry is permanently mounted in a main housing of the breathalyser and wherein at least the sensor and the second electronic circuitry form part of a module which is removably mountable in or on the main housing and wherein the second electronic circuitry is removably connectable to the first electronic circuitry.

2. The breathalyser as claimed in claim 1 wherein the first electronic circuitry comprises a first electrical connector part and the second electronic circuitry comprises a second electrical connector part which is removably connectable to the first electrical connector part.

3. The breathalyser as claimed in claim 2 wherein the first electronic circuitry is provided on a first printed circuit board which is permanently mounted in the main housing and wherein the module comprises a second printed circuit board for the second electronic circuitry, wherein the second printed circuit board is removably receivable in the main housing and wherein the second electronic circuitry is removably connectable to the first electronic circuitry via the first and second electrical connector parts.

4. The breathalyser as claimed in claim 2 wherein the module is in the form of a cartridge comprising a cartridge housing carrying the breath chamber, the sensor, the fluid moving means, the second electronic circuitry comprising the memory arrangement and the second electrical connector part, wherein the cartridge housing is one of a) removably receivable in a slot defined in the main housing and b) removably attachable to the main housing in piggyback fashion and wherein the second electronic circuitry is removably connectable to the first electronic circuitry via the first and second electrical connector parts.

5. The breathalyser as claimed in claim 1 wherein the fluid moving means comprises a bellows which is operable by a solenoid which is controlled by the controller.

6. The breathalyser as claimed in claim 1 wherein the memory arrangement of the second electronic circuitry is a secure memory arrangement enabling confidential non-volatile data storage.

7. The breathalyser as claimed in claim 1 wherein the fuel cell comprises a fuel cell housing and wherein a temperature adjustment means is provided adjacent at least part of the fuel cell housing.

8. The breathalyser as claimed in claim 7 wherein a remainder of the fuel cell housing is cladded by a thermal insulating layer.

9. The breathalyser as claimed in claim 7 comprising a means for detecting motion of the breathalyser, wherein said means is connected to the controller of the first electronic circuitry, wherein the temperature adjustment means comprises and electrically operable heater element which is connected to the controller to be controlled thereby and wherein the controller is configured to cause energization of the heater element upon detection of motion of the breathalyser.

10. The breathalyser as claimed in claim 1 wherein the electronic signal is in the form of a curve, which after excitation of the fuel cell by a breath sample, reaches a peak value and then decays to a base line value and wherein the curve has at least some of the following parameters: i) a peak response value; ii) a peak response time, which is the time from excitation of the fuel cell to when the peak response is reached; iii) an entire response time, which is the time from excitation to when the signal again reaches the base line; and iv) a surface area under the curve, wherein at least some of said parameters vary over time as a function of number of excitations and wherein the memory arrangement of the second electronic circuitry is configured to store: a) pre-derived profile data, as a function of number of excitations, relating to at least some of the parameters; and b) a counter for a value for the number of past excitations of the fuel cell, in use.

11. The breathalyser as claimed in claim 1 wherein the electronic signal is in the form of a curve, which after excitation of the fuel cell by a breath sample, reaches a peak value and then decays to a base line value and wherein the controller of the first electronic circuitry is configured to process the signal to derive data relating to electrical charge which is caused by each excitation of the fuel cell and to store the data in the memory arrangement of the second electronic circuitry.

12. A breathalyser as claimed in claim 11 wherein the processing comprises taking samples of the curved signal over time, determining a mathematical integral of the signal and converting the integral to data relating to electrical charge in coulomb.

13. A breathalyser as claimed in claim 10 wherein the intoxicating substance is alcohol, wherein the sensor comprises an alcohol sensor, wherein the concentration of the intoxicating substance is breath alcohol concentration (BrAC) and wherein an algorithm executed by the processor of the controller of the first electronic circuitry utilizes the value in the counter and the pre-derived profile data to generate BrAC data for the sample, thereby taking into account variations in the parameters with number of excitations.

14. The breathalyser as claimed in claim 13 wherein the BrAC data for each sample and associated time and date data are stored by the controller in the secure memory arrangement.

15. A breathalyser as claimed in claim 1 wherein the intoxicating substance is marijuana and wherein the electronic signal at the output of the sensor is representative of a concentration of tetrahydrocannabinol (THC) in the breath sample.

16. A breathalyser as claimed in claim 15 wherein the sensor comprises carbon nanotubes.

17. A replaceable module for a breathalyser comprising a main housing, the main housing accommodating at least first electronic circuitry comprising a controller comprising a processor for processing a signal representative of a concentration of an intoxicating substance in a breath sample in a breath chamber of the breathalyser and which signal is received from a sensor for an intoxicating substance which is in fluid flow communication with an outlet of the breath chamber, the processor, in use, generating data relating to the concentration of the intoxicating substance, the module comprising at least:
the sensor; and
second electronic circuitry comprising a memory arrangement;
wherein the module is removably mountable in or on the main housing and wherein the second electronic circuitry is removably connectable to the first electronic circuitry.

18. The replaceable module as claimed in claim 17 which is in the form of a cartridge comprising a cartridge body, the cartridge body carrying at least the breath chamber defining an inlet for the breath sample and an outlet; the sensor; fluid moving means for moving the sample via the outlet to the sensor; and wherein the second electronic circuitry comprises a second connector part which is presented on an outside of the cartridge body, wherein the cartridge body is one of a) removably receivable in a slot defined in the main housing and b) removably attachable to the main housing in piggyback fashion and wherein the second electronic circuitry is removably connectable to the first electronic circuitry via the second electrical connector part engaging a first electrical connector part of the first electronic circuitry on the main housing.
